(19) United States
(12) Patent Application Publication
(10) Pub. No.: US 2026/0360511 A1
(43) Pub. Date: Nov. 12, 2026

(54) SYSTEMS AND METHODS FOR EXPLAINABLE, PROGNOSTIC-INFORMED ARTIFICIAL INTELLIGENCE IN CLINICIAN-GOVERNED MENTAL AND BEHAVIORAL HEALTH DECISION SUPPORT WITH ETHICAL CONSTRAINT ENFORCEMENT AND EVIDENTIARY AUDITABILITY

(71) Applicant: Richard Joseph Mitchell, Tavares, FL (US)

(72) Inventor: Richard Joseph Mitchell, Tavares, FL (US)

(21) Appl. No.: 19/575,876

(22) Filed: Mar. 23, 2026

(51) Int. Cl. G16H 20/70

(52) U.S. Cl. CPC G16H 20/70

(57) ABSTRACT

An explainable, prognostic-informed artificial intelligence system provides clinician-governed decision support in mental and behavioral health, extending the AURA platform. Data sources—including conversational signals from language model-mediated patient interactions—are monitored for reliability, temporal validity, and bias drift using modality-specific exponential decay with prognostic remaining-useful-life estimates. An adaptive inference engine employs Dempster-Shafer evidential reasoning separating uncertainty from conflict. An ethical constraint engine applies hybrid utilitarian-deontological optimization with configurable vulnerability hierarchies, governing language model outputs through real-time conversational safety constraints. A tiered clinician governance model dynamically adjusts operational constraints across active supervision, on-call, and constrained autonomous safety modes with monotonically conservative thresholds. Distributional drift detection gates unsafe outputs. An audit subsystem cryptographically binds all evidentiary artifacts in a hash-chained Merkle Tree log within a Trusted Execution Environment. Veteran-specific embodiments incorporate military population vulnerability hierarchies, temporal risk adjustment, and integration with veteran health care systems.

### CROSS-REFERENCE TO RELATED APPLICATIONS

This application is related to the following co-pending applications, commonly owned by the same assignee: “System and Method for a Unified, Evidentiary Artificial Intelligence Architecture” (the “SPPPA Application,” application Ser. No. 19/371,481, filed Oct. 28, 2025); “Redundant Control Architecture for Multi-Domain Autonomous Agents” (the “HRFSA Application,” application Ser. No. 19/371,989, filed Oct. 28, 2025); “Autonomous and Cooperative Multi-Agent Systems for Predictive and Co-Evolutionary Threat Management” (the “ACUS Application,” application Ser. No. 19/373,566, filed Oct. 29, 2025); and “System And Method For Verifiable, Ethical Arbitration and Immutable Auditing Of Autonomous Decisions Using Constrained Execution Environments” (the “Ethical Governance Application,” application Ser. No. 19/383,681, filed Nov. 9, 2025). The disclosures of the above-referenced applications are incorporated herein by reference in their entirety to the extent not inconsistent with the present disclosure. The present application does not claim priority to any of the above-referenced applications.

The present application discloses a domain-specific artificial intelligence architecture for clinical mental and behavioral health decision support within the AURA (Autonomous Unified Reliability Architecture) platform. The architecture incorporates and adapts concepts including a Hybrid Redundant Fail-Safe Architecture (HRFSA) with prognostics and health management, adaptive data fusion, evidentiary audit, and a Hierarchical Constraint Logic Processor (HCLP), with psychology-specific adaptations including ethical constraint enforcement using hybrid ethical theories, user-configurable vulnerability hierarchies, evidential reasoning with explicit conflict and uncertainty separation, and temporal validity modeling with modality-specific decay functions.

### BACKGROUND OF THE INVENTION

### Technical Field

The present invention relates generally to computer-implemented artificial intelligence systems operating in regulated, human-critical domains. More particularly, the invention relates to explainable, prognostic-informed, clinician-governed artificial intelligence systems that provide decision support in mental health, behavioral health, psychology, and psychiatry while preserving professional judgment, ethical safeguards (including beneficence, non-maleficence, autonomy, justice, and explicability per AI4People framework), and evidentiary auditability. This encompasses adaptations for high-stakes environments where data degradation, bias, distributional drift, or evidential conflict could lead to clinical harm, ensuring proactive management aligned with the IEACP (Identification, Estimation, Analysis, Communication, Planning) stages of clinical decision-making.

### Background Art

Mental and behavioral health practice relies on heterogeneous, subjective, and longitudinal data sources, including patient-reported outcomes, clinician observations, structured psychological instruments (e.g., DSM-5 aligned assessments), contextual or behavioral signals from wearable devices, and longitudinal clinical histories. Such data sources vary significantly in reliability, completeness, bias, and temporal relevance, with risks amplified by cognitive biases, emotional volatility, reporting fatigue, or contextual shifts.

Conventional artificial intelligence systems applied to mental health suffer from two critical technical deficiencies. First, they assume static validity of psychological inputs, weighting old data equally with recent data even though clinical data degrades at different rates depending on the modality—acute mood state data may become unreliable within hours, whereas diagnostic history remains relevant for years. Second, they perform conflict masking, whereby contradictory signals (e.g., a verbal denial of distress vs. physiological indicators of acute autonomic arousal) are averaged out or suppressed rather than preserved and elevated as a clinical signal for the governing clinician.

Furthermore, existing systems fail to explicitly represent and separate uncertainty (lack of information) from conflict (contradictory information), creating over-confident outputs when the system should instead acknowledge ambiguity. In clinical psychology, this distinction is safety-critical: uncertainty should lead to broader data collection, whereas conflict should trigger immediate clinician review.

Regulatory and professional standards increasingly require human-supervised, auditable decision-support tools that explicitly manage uncertainty and preserve clinician accountability. The Health Insurance Portability and Accountability Act (HIPAA), the American Psychological Association (APA) Ethical Principles, and global standards including the EU AI Act mandate transparent, auditable, and human-governed AI systems in clinical contexts. Existing AI decision support tools in mental health lack the evidentiary audit trails, cryptographic integrity assurance, and ethical constraint enforcement mechanisms necessary to satisfy these requirements.

The co-pending AURA applications disclose a comprehensive architecture for proactive fault management in multi-domain autonomous agents. Specifically, the HRFSA Application discloses a Prognostics and Health Management (PHM) module that monitors sensor health via Statistical Process Control (SPC) algorithms, generates structured prognostic fault signatures including Remaining Useful Life (RUL), and proactively inflates measurement-noise covariance in a Kalman-type estimator before data corruption occurs. The SPPPA Application discloses a Temporal Block Sparse Attention (TBSA) mechanism for efficient long-range dependency analysis, a synchronic correlation engine for time-indexed document retrieval, and a cryptographic Evidentiary Analyze State with hash-chained immutability. The Ethical Governance Application discloses a Hierarchical Constraint Logic Processor (HCLP) executing within a Trusted Execution Environment (TEE) with Physically Unclonable Function (PUF) attestation, applying Required Constraints (non-negotiable safety) and Preferential Constraints (ethical mandates with harm scores) while logging rejected alternatives with counterfactual harm scores in a Merkle Tree audit structure.

However, the co-pending AURA applications do not fully adapt these capabilities to the unique characteristics of mental health data and clinical psychology practice. Mental health data exhibits domain-specific degradation modes—semantic inconsistency in patient reports, bias drift in behavioral signals across demographic populations, and evidential conflict between subjective and objective data modalities—that differ fundamentally from the sensor degradation and hardware faults addressed in those applications. Moreover, the ethical constraints in clinical psychology (beneficence, non-maleficence, patient autonomy, distributive justice) require a different constraint formulation than the physical safety and preservation hierarchies disclosed in the Ethical Governance Application.

The challenge of providing adequate mental health support during periods when clinical staff are unavailable is particularly acute in military and veteran populations. Epidemiological research has demonstrated that the peak adjusted prevalence of veteran suicide occurs between the hours of 0000 and 0300, when clinical help is typically unavailable, with a Standardized Incidence Ratio of 8.17 at midnight (95% CI: 7.45-8.94) after adjusting for the population awake. This temporal pattern indicates that veterans are eight times more likely to die by suicide at midnight than expected given the population awake, creating a critical need for technology-enabled support that can operate safely during off-hours while maintaining appropriate clinical governance.

Recent advances in large language models (LLMs) have demonstrated the feasibility of conversational artificial intelligence systems that can engage in naturalistic dialogue on mental health topics. However, the deployment of such systems in clinical contexts—particularly with populations at acute risk of self-harm—introduces safety-critical requirements that current LLM architectures do not inherently satisfy. Specifically, language models are known to generate outputs that are fluent but clinically inappropriate, factually incorrect, or inconsistent with established therapeutic protocols. Without an external governance architecture that monitors, constrains, and audits the model's conversational outputs in real time, a conversational clinical co-pilot poses risks including: provision of clinically harmful advice; failure to detect or appropriately escalate acute crisis indicators within conversational context; generation of responses that violate patient autonomy, confidentiality, or therapeutic boundaries; and production of outputs derived from training distributions that do not adequately represent the veteran population, including combat-related post-traumatic stress disorder (PTSD), moral injury, military sexual trauma (MST), and service-connected traumatic brain injury (TBI).

Furthermore, existing conversational AI systems in mental health operate in a binary governance mode—either fully supervised by a clinician or fully autonomous—failing to accommodate the operational reality that clinical supervision availability varies across a continuum. There exists a need for a tiered governance architecture that dynamically adjusts the system's operational autonomy and safety constraints based on the current availability of clinical oversight, enabling safe off-hours operation without compromising clinical accountability.

Accordingly, there exists a need for artificial intelligence systems that: provide decision support rather than autonomous diagnosis; dynamically manage data reliability, temporal validity, and evidential conflict using prognostic monitoring with modality-specific decay functions; explicitly separate uncertainty from contradiction using evidential reasoning frameworks; generate explainable reasoning artifacts with multimodal outputs for clinical accessibility; preserve clinician authority and accountability through governance interfaces; enforce ethical constraints via hybrid utilitarian and deontological theories with user-configurable vulnerability hierarchies; detect distributional drift to prevent unsafe algorithmic extrapolation; and produce auditable evidentiary records with cryptographic integrity suitable for evaluation, governance, and regulatory compliance.

The present invention provides specific and concrete technical improvements to computer-implemented clinical inference systems. The prior art treats clinical AI inference as a statistical estimation problem in which contradictory inputs are averaged, suppressed, or discarded. The present invention improves upon this by implementing Proportional Conflict Redistribution (PCR) to preserve contradictory signals as a quantitative first-class output—a specific mathematical operation on the mass function conflict term k=Σm₁ (A)·m₂ (B) for all A N B=Ø that no human clinician performs in their natural reasoning process. The modality-specific exponential decay function Vᵢ (t)=e\hat{ }(−λᵢ·(t−tᵢ) is not a mental process but a specific computed transformation applied to every data element at every inference cycle, enabling the inference engine to detect and quantify data staleness continuously and automatically. The hash-chain formula Hₙ=Hash(Dataₙ∥Signature_Clinician∥Hₙ₋₁) is a specific cryptographic binding operation that irrevocably links the clinician's identity and decision to the AI output at the hash level, providing forensic properties that no paper-based or manual audit system can achieve. These are improvements to the technical functioning of the computer-implemented inference and audit architecture, not merely the automation of mental processes or the application of abstract mathematical relationships.

### BRIEF SUMMARY OF THE INVENTION

The present invention provides an explainable, prognostic-informed artificial intelligence architecture configured for clinician-governed decision support in mental and behavioral health contexts, incorporating and adapting the AURA HRFSA with domain-specific adaptations for clinical data management, evidential reasoning, ethical constraint enforcement, and evidentiary auditability.

In one embodiment, heterogeneous psychological and behavioral data are ingested from multiple data sources. A Data Health Monitoring Module evaluates the reliability, uncertainty, temporal validity, and bias drift associated with each data source and generates a structured confidence signature. The temporal validity of each data element is assessed using an exponential decay model Vᵢ (t)=e\\hat{ }(−λᵢ·(t−tᵢ)) where λᵢ is a decay constant specific to the clinical modality and tᵢ is the acquisition time, allowing the system to naturally down-weight stale observations while preserving long-term clinical context. Each confidence signature incorporates a prognostic estimate including a Remaining Useful Life (RUL) for data validity, analogous to the HRFSA's prognostic fault signature.

An Adaptive Inference Engine generates decision-support outputs by dynamically weighting the data sources as a function of their confidence signatures. The inference engine explicitly separates uncertainty (lack of information) from conflict (contradictory information) using an evidential reasoning framework such as Dempster-Shafer Theory (DST). When a data source exhibits low temporal validity or high ambiguity, the system redistributes belief mass to an explicit uncertainty state (⊖) rather than assigning it to a negative outcome. Contradictory signals between data modalities are preserved using conflict redistribution logic such as Proportional Conflict Redistribution (PCR), elevating the conflict as a clinical signal rather than masking it through averaging.

An Ethical Constraint Engine models decisions as constrained optimizations using hybrid ethical theories, combining utilitarian harm minimization with deontological rule adherence. The engine applies user-configurable vulnerability hierarchies (e.g., acute crisis prioritized over routine monitoring) and gates or escalates outputs when predefined uncertainty, risk, or distributional drift thresholds are exceeded. Distributional drift is monitored using information-theoretic metrics such as Kullback-Leibler (KL) Divergence, with safety gating triggered upon exceedance of a Population Stability Index (PSI) threshold.

An Explanation Generation Module produces human- and machine-readable reasoning traces identifying contributing data sources, relative weighting, uncertainty intervals, conflict signals, and ethical rationale. A Clinician Governance Interface requires explicit clinician approval, modification, or rejection of outputs prior to any clinical use.

An Audit Subsystem records outputs, reasoning traces, confidence signatures, clinician interactions, and ethical evaluations in a tamper-evident, cryptographically verifiable format. In preferred embodiments, this utilizes a hash-chained log or Merkle Tree where each record cryptographically binds the AI output, the clinician's governance signature, and the preceding record's hash (H_n=Hash(Data_n\|\|Signature_Clinician \|\|H\_{n−1})), ensuring cryptographic non-repudiation of clinician governance actions, wherein each audit record irrevocably binds the clinician's identity and decision to the specific AI output, and support for IEACP-compliant review processes.

The system is expressly configured as decision support only and does not autonomously diagnose mental disorders, prescribe treatment, or initiate clinical actions, ensuring alignment with non-maleficence and patient autonomy.

In certain embodiments, the system is configured to govern a conversational language model that generates candidate responses for patient interaction in mental or behavioral health contexts. The system evaluates each candidate response through the Adaptive Inference Engine and Ethical Constraint Engine prior to transmission, suppressing clinically inappropriate responses and replacing them with safe defaults from a pre-approved response library. A tiered clinician governance model dynamically adjusts the system's operational constraints across active supervision, on-call, and constrained autonomous safety modes, with monotonically conservative safety thresholds ensuring that reduced clinical oversight triggers increased system conservatism. In veteran-specific embodiments, the system incorporates military population vulnerability hierarchies, temporal risk adjustment informed by suicide epidemiology, and integration with veteran health care information systems and crisis services.

### BRIEF DESCRIPTION OF THE SEVERAL VIEWS OF THE DRAWINGS

FIG. 1 is a block diagram illustrating an overall system architecture for clinician-governed mental and behavioral health decision support, showing the seven core modules and their data flow relationships.

FIG. 2 is a block diagram illustrating the Data Health Monitoring Module (200), including temporal validity modeling with modality-specific exponential decay, bias drift detection, and structured confidence signature generation.

FIG. 3 is a block diagram illustrating the Adaptive Inference Engine (300), including evidential reasoning with Dempster-Shafer belief mass redistribution, Proportional Conflict Redistribution, and confidence-conditioned dynamic weighting.

FIG. 4 is a block diagram illustrating the Explanation and Provenance Generation Subsystem (400), including reasoning trace composition, conflict signaling, and multimodal output generation.

FIG. 5 is a block diagram illustrating the Clinician Governance Interface (500), including approval workflows, rationale capture, and user-configurable vulnerability hierarchies.

FIG. 6 is a block diagram illustrating the Audit and Evidentiary Logging Subsystem (600), including hash-chained log structure, synchronic correlation for regulation-versioned audit, and IEACP stage tracking.

FIG. 7 is a block diagram illustrating the Ethical Constraint Engine (700), including hybrid utilitarian-deontological constrained optimization, distributional drift detection via KL Divergence, and safety gating logic.

FIG. 8 is a data flow diagram illustrating the integration of the present system with the broader AURA architecture, including Trusted Execution Environment (TEE) boundaries, PUF-derived attestation flows, and TBSA-enabled clinical LLM processing.

FIG. 9 is a data flow diagram illustrating a conversational language model governance architecture, showing the relationship between a language model response generator, a response-level safety evaluation by the Adaptive Inference Engine (300), real-time ethical constraint filtering by the Ethical Constraint Engine (700), a safe default response library, conversational signal ingestion by the Data Health Monitoring Module (200), and a conversational audit trail maintained by the Audit Subsystem (600).

FIG. 10 is a state transition diagram illustrating a tiered clinician governance model, showing three governance tiers (Tier 1: Active Supervision, Tier 2: On-Call Supervision, Tier 3: Constrained Autonomous Operation), authenticated clinician actions governing tier transitions, tier-specific safety threshold configurations, and the monotonically conservative constraint relationship between tiers.

### DETAILED DESCRIPTION OF THE INVENTION

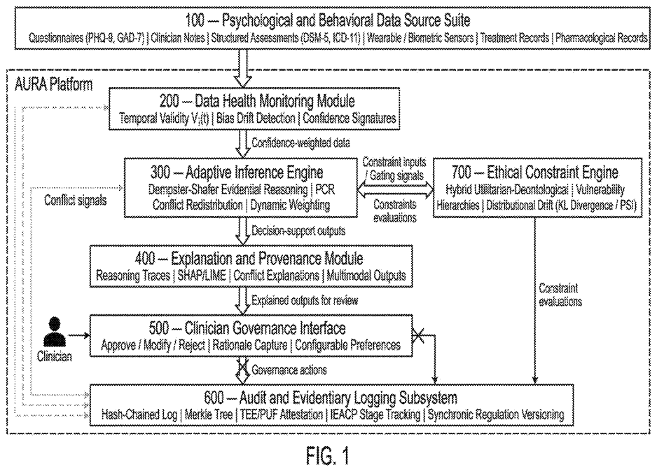

### I. System Overview (FIG. 1)

The system comprises seven cooperating modules that collectively provide explainable, uncertainty-aware, clinician-governed decision support. These modules adapt the AURA platform's proactive fault compensation architecture to the domain of clinical mental and behavioral health:

TABLE 1

| | | | |
|---|---|---|---|
| 200 | Data Health | HRFSA Prognostics and | Management (PHM) |
| | Monitoring | Health Module | Module |
| 300 | Adaptive | Adaptive Hybrid Data | |
| | Inference Engine | Fusion Module | |
| | HRFSA | | |
| 400 | Explanation and | HCLP Counterfactual | |
| | SPPPA | Log | |
| | Evidentiary | | |
| | Analyze State/ | | |
| | Provenance | | |
| | Module | | |
| 500 | Clinician | Novel (domain-specific | |
| | Governance | Interface human-in- | |
| | | the-loop) | |
| 600 | Audit and | SPPPA Cryptographic | Management/Merkle |
| | Evidentiary | State Logging | Tree Audit |
| | | Subsystem | |
| 700 | Ethical Constraint | HCLP Required + | |
| | Engine | Preferential Constraints | |

These components operate cooperatively to implement a clinical decision support pipeline in which: raw clinical data is continuously monitored for reliability and validity (200); monitored data is fused into probabilistic decision-support outputs using evidential reasoning conditioned on data quality (300); outputs are subject to ethical constraint evaluation and safety gating (700); approved outputs are presented with full explanatory provenance (400); a clinician must explicitly approve, modify, or reject each output (500); and all system states, reasoning traces, and governance actions are cryptographically recorded (600). No output reaches clinical use without traversing this complete pipeline.

### II\. Psychological and Behavioral Data Sources (100)

The data source suite (100) ingests data from multiple heterogeneous clinical sources, including without limitation: patient-reported questionnaires and outcome measures (e.g., PHQ-9 for depression, GAD-7 for anxiety); clinician notes and observational records; structured psychological assessments aligned with recognized diagnostic frameworks (e.g., DSM-5, ICD-11); behavioral or contextual signals acquired from wearable devices, digital phenotyping applications, or environmental sensors; longitudinal clinical history, treatment records, and prior diagnoses; and pharmacological records and medication adherence data.

Each data source is treated as a monitored input subject to continuous reliability assessment—not as assumed ground truth. The system treats each source as analogous to a sensor in the HRFSA disclosed in the co-pending HRFSA Application, applying the same philosophy of proactive degradation management but adapted for the stochastic, subjective, and temporally varying characteristics of clinical psychological data.

In certain embodiments, the data source suite includes preprocessing for ethical equity, including detection of underrepresentation in training data for specific demographic populations, assessment of cultural context factors that may affect data interpretation, and flagging of data collection conditions that may introduce systematic bias (e.g., assessments administered during high-stress institutional contexts).

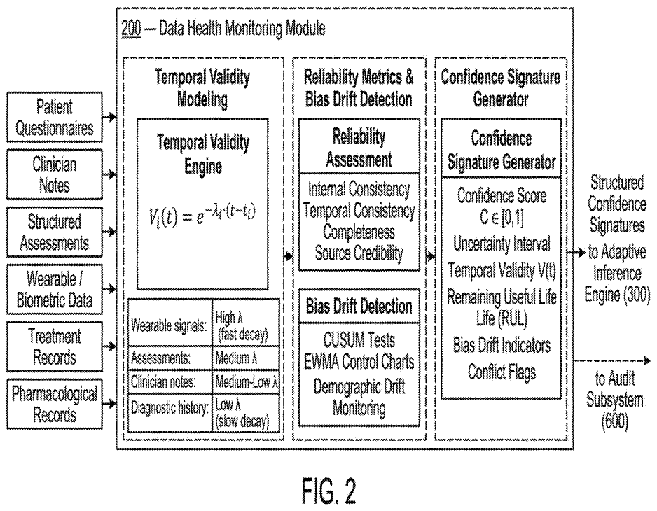

### III\. Data Health Monitoring and Confidence Signatures (200) (FIG. 2)

The Data Health Monitoring Module (200) continuously evaluates each data source for reliability metrics and generates a structured confidence signature encoding the current reliability state. This module adapts the HRFSA's Prognostics and Health Management (PHM) module from hardware sensor monitoring to clinical data source monitoring, applying the same fundamental principle: detect incipient degradation proactively and generate structured prognostic information before degraded data corrupts downstream inference.

### A. Temporal Validity Modeling

The system calculates a dynamic validity score V(t) for every data element. Unlike binary “valid/invalid” flags used in conventional clinical AI systems, the present invention applies an exponential decay model where the decay constant is specific to the clinical modality:

Vᵢ(t)=e\\hat{ }(−λᵢ·(t−tᵢ))

where tᵢ is the acquisition time of data element i, λᵢ is the modality-specific decay constant for data source i, and t is the current evaluation time. The term (t−tᵢ) represents the elapsed time since acquisition, anchoring the decay to the specific moment the data was collected. The modality-specific decay constant reflects the clinical reality that different types of psychological data lose relevance at different rates.

TABLE 2

| | | |
|---|---|---|
| Behavioral signal | Medium-High λ | Behavioral patterns shift with |
| | | (wearable) context; sleep or activity |
| | | data degrades over days |
| Structured assessment score | Medium λ | Standardized instruments retain |
| | | validity for weeks but not indefinitely |
| Clinician observational note | Medium-Low λ | Professional observations retain |
| | | relevance for weeks to months |
| Diagnostic history/trauma | Low λ | Historical diagnoses and trauma |
| record | | records remain relevant for |
| | | years |
| Pharmacological record | Context-dependent λ | Validity tied to medication |
| | | adherence; decays rapidly upon |
| | | suspected non-adherence |

This exponential decay model allows the Adaptive Inference Engine to naturally down-weight stale observations while maintaining long-term clinical context, avoiding both the static validity assumption of prior art and the binary discard approach that loses potentially relevant longitudinal information.

### B. Reliability Metrics and Bias Drift Detection

In addition to temporal validity, the Data Health Monitoring Module evaluates each data source for one or more of the following reliability metrics: internal consistency (e.g., contradiction between items within a self-report instrument); temporal consistency (abrupt changes inconsistent with clinical trajectory); completeness (missing data rates); bias drift (systematic shifts in data distribution relative to training population, detected using statistical monitoring analogous to the HRFSA's SPC-based fault detection as disclosed in the co-pending HRFSA Application); and source credibility indicators (e.g., device calibration status for wearables, response pattern analysis for self-reports).

Bias drift is monitored as a distinct clinical safety signal. In preferred embodiments, the module applies statistical process control techniques adapted from the HRFSA—including Cumulative Sum (CUSUM) tests and Exponentially Weighted Moving Average (EWMA) control charts—to detect systematic shifts in data source behavior that may indicate demographic bias, instrument degradation, or environmental confounds. When bias drift is detected, it is encoded within the structured confidence signature and elevated for clinician and ethical review.

### C. Structured Confidence Signature

Based on the temporal validity score and reliability metrics, the module generates a structured confidence signature for each data source. The confidence signature is a structured data object comprising at least: a quantitative confidence score C∈\[0, 1\]; an uncertainty interval; a temporal validity score V(t) as computed above; a prognostic estimate including a Remaining Useful Life (RUL) for data validity, indicating the estimated time until the data source's reliability falls below a clinically useful threshold; one or more bias drift indicators; and one or more conflict flags indicating contradiction with other data sources.

The structured confidence signature is the clinical analog of the HRFSA's structured prognostic fault signature (comprising source ID, fault type, confidence score, and RUL/_{T}). Just as the HRFSA's fault signature drives proactive reconfiguration of the Kalman estimator's measurement-noise covariance, the confidence signature drives proactive reconfiguration of the Adaptive Inference Engine's evidential weighting.

The Remaining Useful Life (RUL) for data validity is defined in the present system as the estimated elapsed time from the current evaluation moment until the validity score Vᵢ(t) for data source i falls below a clinically significant threshold V_min. This threshold represents the minimum validity score at which the data source's contribution to inference remains clinically meaningful. Formally, RULᵢ(t)=(−(1/λᵢ)·In(V_min))−(t−tᵢ), where V_min is a configurable parameter stored in the governance configuration record for each data modality. Default values for V_min reflect clinical convention: V_min=0.50 for behavioral signals from wearable devices (meaning the data is considered clinically relevant until its validity falls below 50% of initial value); V_min=0.25 for structured assessment scores; V_min=0.10 for diagnostic history and trauma records (reflecting their long-term clinical relevance). When a data source's RUL falls below a warning threshold (default: 10% of the modality's expected useful life), the system generates a data refresh advisory in the reasoning trace and confidence signature, prompting the governing clinician to consider collecting updated data. When RUL reaches zero, the data source's belief mass is fully transferred to the uncertainty state ⊖ in the DST framework, effectively excluding the source from contributing to specific clinical hypotheses while preserving the audit record of its prior contributions.

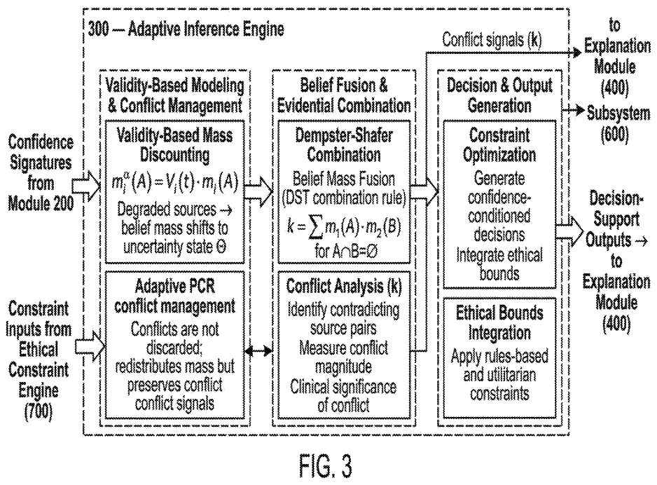

### IV\. Adaptive Inference Engine (300) (FIG. 3)

The Adaptive Inference Engine (300) generates decision-support outputs by dynamically adjusting the evidential influence of each data source based on its corresponding confidence signature. This module adapts the HRFSA's Adaptive Hybrid Data Fusion Module from Kalman-type estimation to evidential reasoning, adapted for the fundamentally different data characteristics of clinical psychology.

### A. Evidential Reasoning Framework

In a preferred embodiment, the Adaptive Inference Engine utilizes an evidential reasoning framework based on Dempster-Shafer Theory (DST) or functionally equivalent belief function frameworks. The critical technical advantage of evidential reasoning over conventional probabilistic fusion (e.g., Bayesian inference or weighted averaging) is the explicit separation of uncertainty from conflict—a distinction that is safety-critical in clinical mental health.

Under the DST framework, each data source provides a mass function mᵢ(·) over a frame of discernment Q representing the set of possible clinical hypotheses. When a data source exhibits low temporal validity V(t) or high ambiguity as indicated by its confidence signature, the system redistributes belief mass to the explicit uncertainty state ⊖ (the full frame of discernment) rather than assigning it to any specific clinical hypothesis. This prevents the system from generating over-confident outputs based on degraded or ambiguous data—a direct clinical adaptation of the HRFSA's principle of proactively reducing the influence of degraded inputs before they corrupt the state estimate.

Formally, given a confidence signature with temporal validity Vᵢ(t) for source i, the system applies a validity-based discounting operation to the mass function before combination. For each focal element A ⊂Ω representing a specific clinical hypothesis:

mᵢ^{a}(A)=Vᵢ(t)·mᵢ(A)

mᵢ^{a}(⊖)=1−Vᵢ(t)+Vᵢ(t)·mᵢ(⊖)

where mᵢ^{a} is the discounted mass function and ⊖ is the full frame of discernment representing total ignorance. This formulation ensures that as a data source's temporal validity decays, its belief mass is progressively transferred to the ignorance state rather than to any specific clinical hypothesis. This prevents the system from generating over-confident outputs based on degraded or ambiguous data—a direct clinical adaptation of the HRFSA's principle of proactively reducing the influence of degraded inputs before they corrupt the state estimate.

### B. Conflict Preservation via Proportional Conflict Redistribution

A critical technical improvement of the present system is the preservation of contradictory signals as a clinical signal rather than masking them through conventional Dempster combination. When data sources provide conflicting evidence—for example, a patient verbally denying suicidal ideation while biometric sensors indicate acute autonomic arousal (elevated heart rate, galvanic skin response, respiratory rate changes)—the system must not average or suppress this contradiction.

In a preferred embodiment, the system employs Proportional Conflict Redistribution (PCR) rules (e.g., PCR5 or PCR6) to handle inter-source conflict. Under PCR, conflicting belief mass is not discarded or normalized away (as in classical Dempster's rule of combination) but instead redistributed proportionally to the sources involved in the conflict, preserving a quantitative measure of the conflict magnitude. The conflict mass k=Σm₁ (A)·m₂ (B) for all A ∩B=Ø is computed and reported as a first-class clinical signal in the system's output.

This conflict signal is transmitted to the Explanation Module (400) for inclusion in the reasoning trace and to the Ethical Constraint Engine (700) for risk evaluation. High conflict values trigger escalation to the Clinician Governance Interface (500), ensuring that clinically significant contradictions are never hidden from the governing clinician.

### C. Dynamic Weighting and Confidence-Conditioned Inference

The inference engine dynamically adjusts data source weighting during ongoing clinical operation. Data sources exhibiting degradation, increased uncertainty, bias drift, or low temporal validity as indicated by their confidence signatures are down-weighted or excluded from inference. The system continuously re-evaluates weighting as new confidence signatures are generated, creating a prognostic-informed inference loop analogous to the HRFSA's continuous Kalman gain adjustment based on PHM fault signatures.

In certain embodiments, the inference engine models its decision-support outputs as constrained optimizations, integrating inputs from the Ethical Constraint Engine (700) as constraints on the optimization. This formulation allows the system to generate outputs that are not merely statistically optimal but also ethically bounded—for example, preferring a recommendation with slightly lower statistical confidence if it better satisfies beneficence or non-maleficence constraints.

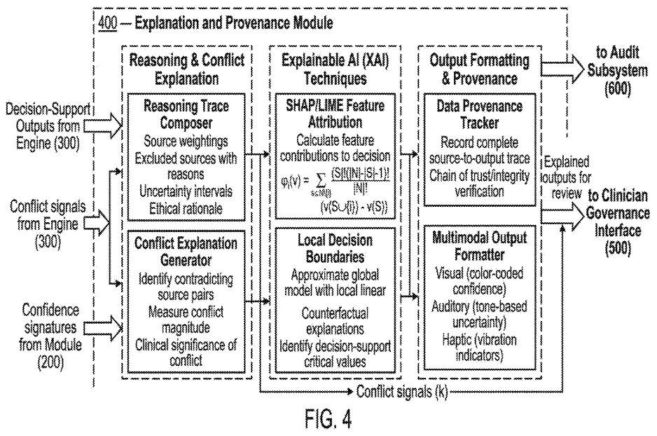

### V. Explanation and Provenance Module (400) (FIG. 4)

The Explanation and Provenance Module (400) generates reasoning traces that provide clinically meaningful transparency into the decision-support output generation process. Each reasoning trace identifies: contributing data sources and their relative weighting in the output; data sources that were down-weighted or excluded due to degraded confidence signatures, with the specific degradation reason; quantitative uncertainty intervals and confidence bounds on the output; conflict signals and the specific data source pairs involved in evidential contradiction; and ethical rationale, including which ethical constraints (if any) affected the output and why vulnerability hierarchies prioritized certain considerations.

In preferred embodiments, the explanation module employs explainable AI (XAI) techniques including SHAP (SHapley Additive explanations) for feature attribution, LIME (Local Interpretable Model-agnostic Explanations) for local decision boundary interpretation, and counterfactual explanations identifying the minimal data changes that would alter the recommendation. These techniques are applied to the evidential reasoning outputs, not merely to a black-box model, providing structured and verifiable explanations.

A particular technical improvement of the present system is the elevation of contradiction as a primary explanation element. When the system detects evidential conflict (via the PCR-computed conflict mass), the explanation module generates a specific conflict explanation identifying the contradicting sources, the magnitude of conflict, and the clinical significance. This transforms what conventional systems treat as noise (contradictory inputs) into actionable clinical intelligence.

In certain embodiments, the reasoning traces are generated in multimodal formats to support clinical accessibility, including visual representations (color-coded confidence indicators, uncertainty visualizations), auditory indicators (tone-based uncertainty signaling for eyes-free clinical contexts), and haptic feedback channels (vibration-based uncertainty indicators for wearable clinical devices). The multimodal outputs address documented usability challenges in clinical AI adoption, particularly in high-cognitive-load clinical environments where visual attention may be limited.

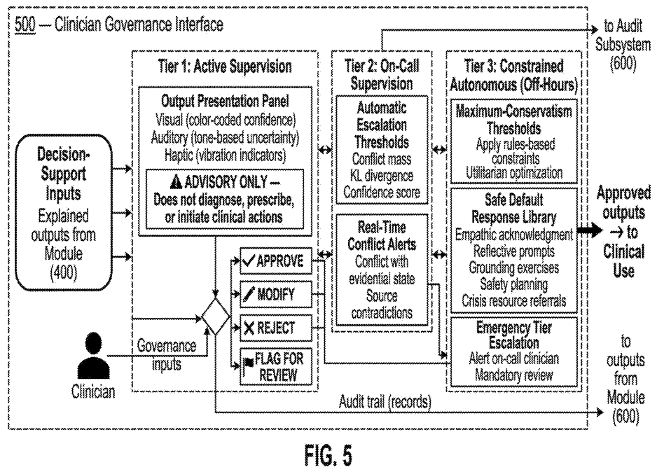

### VI\. Clinician Governance Interface (500) (FIG. 5)

The Clinician Governance Interface (500) presents decision-support outputs and their associated reasoning traces for clinician review. The interface requires explicit clinician action to approve, modify, or reject each output. The system prevents any downstream clinical use of an output absent explicit clinician confirmation, ensuring that the system operates strictly as an advisory tool and never substitutes for professional clinical judgment.

When a clinician modifies or rejects an output, the interface captures the clinician's rationale in a structured format. The modification rationale includes the clinician's assessment of which data sources or considerations the system may have over- or under-weighted, supporting continuous system calibration and retrospective audit.

The governance interface includes user-configurable preferences that allow clinicians to customize the system's behavior within defined safety boundaries. These preferences include vulnerability hierarchy configuration (e.g., prioritizing acute distress indicators over chronic monitoring metrics), explanation verbosity settings, and threshold adjustments for escalation triggers. In certain embodiments, the preferences are synthesized from clinician responses to structured ethical dilemmas (e.g., scenarios balancing patient privacy against intervention urgency), ensuring that the customization reflects the clinician's ethical framework rather than arbitrary parameter adjustment.

The system explicitly labels all outputs as advisory and includes persistent interface elements communicating that the system does not autonomously diagnose mental disorders, prescribe treatment, or initiate clinical actions. This labeling is not merely cosmetic but is enforced architecturally: no output path exists from the inference engine to any clinical action system that does not traverse the clinician governance interface.

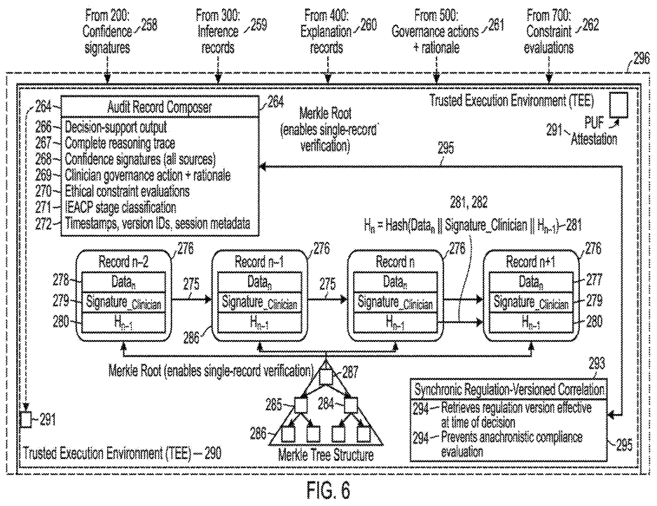

### VII\. Audit and Evidentiary Logging Subsystem (600) (FIG. 6)

The Audit and Evidentiary Logging Subsystem (600) records a comprehensive evidentiary trail of all system operations, adapting the AURA platform's cryptographic audit capabilities to the clinical mental health domain.

### A. Audit Record Content

Each audit record comprises: the decision-support output generated by the Adaptive Inference Engine; the complete reasoning trace generated by the Explanation Module, including data source weightings, uncertainty intervals, and conflict signals; the structured confidence signatures for all data sources at the time of inference; the clinician governance action (approval, modification, or rejection) with captured rationale; ethical constraint evaluations, including which constraints were active, whether gating or escalation occurred, and the ethical rationale; IEACP stage classification, tracking which stage of the clinical decision-making process (Identification, Estimation, Analysis, Communication, Planning) the output supports; timestamps, version identifiers, and session metadata.

### B. Cryptographic Integrity and Non-Repudiation

The audit records are stored in a tamper-evident, cryptographically verifiable format. In a preferred embodiment, the subsystem implements a hash-chained log structure where each record includes a cryptographic hash of the preceding record:

H_n=Hash(Data_n\|\|Signature_Clinician\|\|H\_{n−1})
where H_n is the hash of the current record, Data_n is the serialized content of the current audit entry, Signature_Clinician is the cryptographic signature of the clinician's governance action (approval, modification, or rejection) binding the clinician's identity and decision to the record, and H\_{n−1} is the hash of the immediately preceding record. The inclusion of the clinician's cryptographic signature within the hash input—rather than merely alongside it—ensures that the clinician's governance action is irrevocably bound to the specific AI output and reasoning trace at the cryptographic level, providing forensic non-repudiation that cannot be retroactively altered without invalidating the entire subsequent hash chain. In certain embodiments, the hash chain is anchored into a Merkle Tree structure, enabling efficient verification of any individual record without requiring traversal of the entire chain.

In preferred embodiments integrating with the broader AURA architecture, the audit subsystem executes within a Trusted Execution Environment (TEE) with Physically Unclonable Function (PUF)-derived hardware attestation, as disclosed in the co-pending SPPPA and Ethical Governance Applications. The TEE provides hardware-isolated protection for the audit log generation process, and the PUF provides an intrinsic, decentralized root of trust that cannot be cloned or spoofed, ensuring that the audit records are not merely cryptographically linked but are generated within a verified, tamper-resistant execution environment.

### C. Synchronic Correlation for Regulation-Versioned Audit

In certain embodiments, the audit subsystem incorporates a synchronic correlation capability adapted from the co-pending SPPPA Application. When performing retrospective compliance analysis, the system retrieves the specific version of applicable regulatory rules (e.g., APA Ethical Principles, HIPAA requirements, state-specific mental health statutes, institutional policies) that was in effect at the time of the clinical decision under review. This prevents the anachronistic error of evaluating a past clinical AI-assisted decision against subsequently enacted regulations, providing temporally accurate compliance assessment.

The audit subsystem supports independent evaluation, research replication, and continuous bias auditing. The cryptographic integrity of the audit trail allows third-party evaluators to verify that the system's behavior was consistent with its disclosed operating parameters, supporting regulatory compliance reviews, malpractice litigation defense, and scientific reproducibility of AI-assisted clinical outcomes.

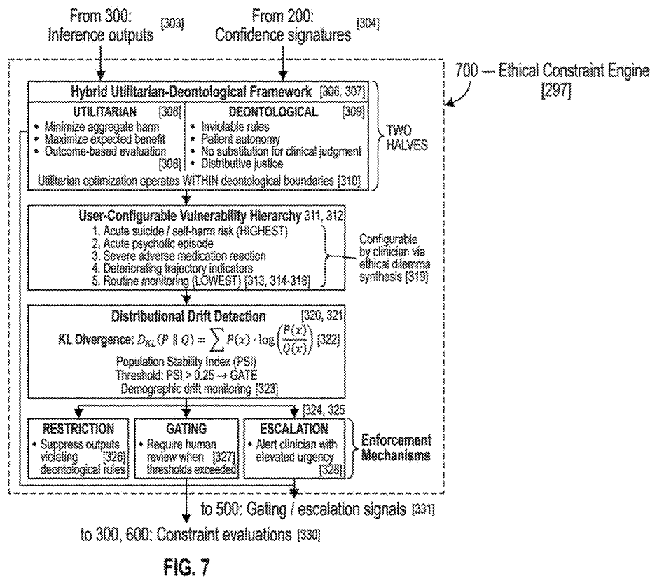

### VIII\. Ethical Constraint Engine (700) (FIG. 7)

The Ethical Constraint Engine (700) constitutes a novel domain-specific adaptation of the AURA platform's Hierarchical Constraint Logic Processor (HCLP). The HCLP as disclosed in the co-pending Ethical Governance Application applies Required Constraints (non-negotiable physical safety parameters) and Preferential Constraints (prioritized ethical mandates with calculated harm scores) in the context of autonomous vehicle collision avoidance, utilizing a fixed preservation hierarchy (human life \>critical infrastructure \>private property). The present Ethical Constraint Engine adapts this architecture for clinical mental health, replacing the physical safety hierarchy with a clinically grounded ethical framework based on hybrid ethical theories.

### A. Hybrid Utilitarian-Deontological Constraint Formulation

The Ethical Constraint Engine models decisions as constrained optimizations that combine two complementary ethical frameworks. The utilitarian component seeks to minimize aggregate harm across the patient population, evaluating decision alternatives based on their expected outcomes and selecting options that maximize expected benefit or minimize expected harm. The deontological component enforces absolute rules that cannot be overridden by utilitarian calculus, including: the system shall never override patient autonomy (e.g., never disclose information without consent, even if disclosure would produce better aggregate outcomes); the system shall never substitute for professional clinical judgment; and the system shall apply consistent standards regardless of patient demographic characteristics (distributive justice).

This hybrid formulation addresses a well-documented limitation of purely utilitarian AI systems in mental health: they may recommend actions that maximize expected outcomes at the expense of individual patient rights. The deontological constraints serve as inviolable boundaries (analogous to the HCLP's Required Constraints) while the utilitarian optimization operates within those boundaries (analogous to the HCLP's Preferential Constraints).

### B. User-Configurable Vulnerability Hierarchies

A technical improvement over the HCLP's fixed preservation hierarchy as disclosed in the co-pending Ethical Governance Application, the Ethical Constraint Engine supports user-configurable vulnerability hierarchies. The clinician may configure the priority ranking of different clinical situations, such as: acute suicide or self-harm risk (highest priority); acute psychotic episode; severe adverse medication reaction; deteriorating trajectory indicators; and routine monitoring and maintenance. The vulnerability hierarchy affects the engine's gating and escalation behavior: when the system detects indicators associated with a high-priority vulnerability category, it applies more conservative safety thresholds and requires faster clinician response.

In certain embodiments, vulnerability hierarchies are synthesized from clinician responses to structured ethical investigation scenarios. The system presents the clinician with a series of clinical dilemmas (e.g., scenarios balancing patient privacy against intervention urgency, or scenarios involving competing obligations between patients in group settings) and derives the clinician's implicit priority structure from their responses. This synthesis ensures that the hierarchy reflects the clinician's professional ethical framework and clinical expertise rather than arbitrary numerical parameter settings.

The synthesis of vulnerability hierarchies from clinician responses employs the following computational procedure: (a) the system presents the clinician with a battery of N structured ethical dilemma scenarios (N≥15 in preferred embodiments, covering the full range of clinical priority categories); (b) for each scenario, the clinician selects a preferred response option, rates the urgency of response on a scale of 1-10, and optionally provides a textual rationale; (c) the system constructs a pairwise priority comparison matrix M∈R\hat{ }(k×k) where M[i,j] represents the number of scenarios in which clinical situation category i was prioritized over category j; (d) an Analytic Hierarchy Process (AHP) or functionally equivalent ranking algorithm derives a normalized priority weight vector w=[w₁, w₂, . . . , wₖ] from M using eigenvector decomposition, with the principal eigenvector of M normalized to sum to 1.0 constituting the vulnerability hierarchy weights; (e) the consistency ratio (CR=CI/RI, where CI is the consistency index and RI is the random index for a k×k matrix) is computed; if CR>0.10, the clinician is notified of inconsistency in their responses and offered the opportunity to revise specific scenario judgments; and (f) the derived weight vector is stored in the governance configuration record and displayed to the clinician for review and confirmation before activation. The complete synthesis session, including each scenario presented, the clinician's response, and the derived weight vector, is recorded in the audit subsystem with the clinician's cryptographic signature, enabling retrospective review of the hierarchy derivation basis.

### C. Distributional Drift Detection and Safety Gating

To prevent unsafe algorithmic extrapolation or the propagation of demographic bias, the Ethical Constraint Engine continuously monitors the statistical distribution of runtime patient data relative to the system's training distribution. This monitoring guards against a specific failure mode in clinical AI: the system producing confident but potentially biased or unreliable outputs when processing data from patient populations or clinical contexts that were underrepresented in training data.

In a preferred embodiment, distributional drift is quantified using Kullback-Leibler (KL) Divergence between the training distribution P and the observed runtime distribution Q:

D\_{KL}(P\|\|Q)=ΣP(x)·log(P(x)/Q(x)

The system aggregates drift measurements into a Population Stability Index (PSI), which provides a scalar indicator of overall distributional shift. When the PSI exceeds a predefined safety threshold (e.g., PSI\>0.25, indicating significant distributional instability), the Ethical Constraint Engine automatically gates the inference output, suppressing or flagging it and requiring manual clinician review before the output may be displayed or acted upon. The PSI threshold is configurable and may be set at different levels for different clinical contexts (e.g., lower thresholds such as PSI \>0.10 for high-acuity crisis assessment, standard thresholds such as PSI \>0.25 for routine progress monitoring).

In certain embodiments, the drift detection module also monitors for specific forms of demographic drift—shifts in the distribution of patient demographic features relative to training demographics—as an additional safeguard against algorithmic bias. When demographic drift is detected, the system logs a bias alert in the audit trail and adjusts the confidence signature of affected outputs to reflect the reduced reliability of inference for the underrepresented population.

### D. Gating, Escalation, and Constraint Enforcement

The Ethical Constraint Engine enforces its constraints through three mechanisms: restriction (suppressing outputs that violate deontological constraints, regardless of utilitarian benefit); gating (requiring human review before displaying outputs when uncertainty, conflict, drift, or risk thresholds are exceeded); and escalation (alerting the clinician with elevated urgency when vulnerability hierarchy triggers or high conflict signals are detected). All instances of restriction, gating, or escalation are recorded in the audit trail with the specific ethical rationale, supporting post-hoc review and continuous improvement.

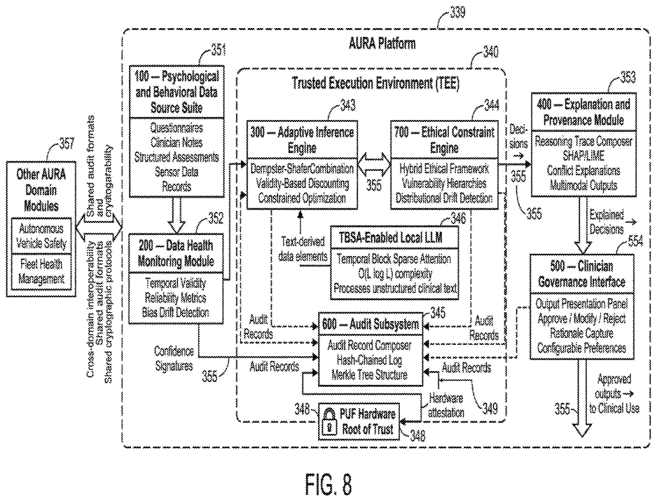

### IX\. AURA Platform Integration (FIG. 8)

In preferred embodiments, the present system is deployed as a module within the broader AURA platform, leveraging shared infrastructure components disclosed in the co-pending applications.

### A. Trusted Execution Environment and Hardware Attestation

The Adaptive Inference Engine (300), Ethical Constraint Engine (700), and Audit Subsystem (600) execute within a Trusted Execution Environment (TEE) as disclosed in the co-pending SPPPA and Ethical Governance Applications. The TEE provides a cryptographically isolated execution environment that protects the inference logic, ethical constraints, and audit log generation from tampering, including by privileged software on the host system. The TEE is anchored by a Physically Unclonable Function (PUF) integrated with the processing device, providing an intrinsic hardware root of trust for remote attestation that verifies the integrity of the execution environment before any clinical inference is performed.

The Trusted Execution Environment (TEE) is a hardware-isolated secure enclave that executes a subset of the system's modules in a cryptographically protected processing environment separated from the main operating system. In a preferred embodiment, the TEE is implemented using Intel Software Guard Extensions (SGX) or ARM TrustZone, providing hardware-enforced memory isolation such that code and data within the enclave cannot be inspected or modified by privileged software outside the enclave, including the host operating system, hypervisor, or other applications. The Physically Unclonable Function (PUF) is a hardware component integrated into the processing device that exploits manufacturing process variations to generate a device-unique, unclonable response to a given challenge input. The PUF provides a hardware root of trust for remote attestation: a verifier can confirm that the TEE is executing on a specific, unmodified hardware device by verifying the PUF-derived attestation response, which cannot be reproduced on any other device even with full knowledge of the device's software configuration. In the present system, the TEE enclave is provisioned at deployment time with the Ethical Constraint Engine's deontological constraint set and the Audit Subsystem's hash-chain signing keys. The PUF attestation verifies the integrity of this provisioning before any clinical inference is permitted. Subsequent remote attestation reports, signed by the PUF-derived key pair, enable the governing clinician or institutional auditor to verify at any time that the system is executing the authorized constraint configuration on the expected hardware, providing a hardware-level complement to the software-layer governance mechanisms described in this application.

### B. Temporal Block Sparse Attention for Clinical Records

In certain embodiments, the Adaptive Inference Engine (300) incorporates a local large language model (LLM) executed within the TEE for processing unstructured clinical text (clinician notes, patient narratives, prior assessment reports). The LLM utilizes the Temporal Block Sparse Attention (TBSA) mechanism disclosed in the co-pending SPPPA Application, which organizes key and value vectors into temporal blocks and processes them via parallel attention paths comprising a sliding window attention path for local context and a compressed token attention path for global context. The TBSA mechanism achieves O(L log L) computational complexity for long-range dependency analysis, enabling efficient processing of longitudinal patient records that may span years of clinical documentation.

The TBSA-enabled LLM processes clinical text in conjunction with the Data Health Monitoring Module (200), which provides temporal validity scores for the text-derived data elements. The LLM's attention mechanism is conditioned on these validity scores, implementing an attention-level analog of the confidence-weighted inference performed at the evidential reasoning level.

### C. Cross-Domain Interoperability

Because the present system is architected as a domain-specific instantiation of the AURA platform, it shares data formats, audit record structures, and cryptographic protocols with other AURA domain instantiations. This enables cross-domain integration scenarios, including: correlation of mental health decision support audit trails with concurrent autonomous system safety records (e.g., for mental health monitoring of autonomous vehicle operators); integration with AURA-based fleet health management for healthcare fleet applications; and aggregated institutional audit reporting across multiple AURA-equipped clinical and operational systems. In FIG. 8, reference numeral 554 designates the explained-decisions data flow path from the Explanation and Provenance Module (400) to the Clinician Governance Interface (500), conveying the fully annotated decision-support outputs, reasoning traces, and conflict signals generated by Module 400 to the governance layer for clinician review prior to clinical use.

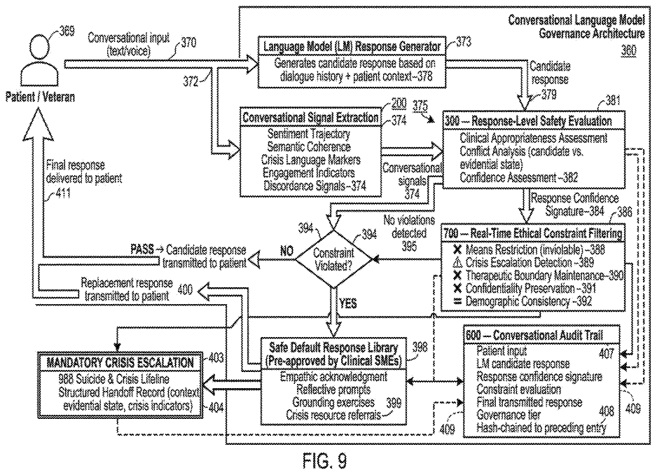

### IX-A. Conversational Language Model Governance Architecture (FIG. 9)

In certain embodiments, the present system is configured to govern a conversational language model (LM) that engages in naturalistic dialogue with patients or clinical support staff in mental health and behavioral health contexts. In this configuration, the language model generates candidate conversational responses based on dialogue history and patient context, and the present system's modules evaluate, constrain, and audit each candidate response before it is transmitted to the patient or user. The language model operates as a data source and response generator within the architecture, while the Adaptive Inference Engine (300), Ethical Constraint Engine (700), and Audit Subsystem (600) collectively function as a real-time governance layer that ensures clinical safety, ethical compliance, and evidentiary auditability of the conversational interaction.

### A. Conversational Signal Ingestion

When the system operates in a conversational governance mode, the Psychological and Behavioral Data Source Suite (100) is extended to ingest conversational signals as additional data sources alongside structured clinical data. Conversational signals include, without limitation: sentiment trajectory across conversational turns, representing the directional change in expressed affect over the course of the dialogue; semantic coherence metrics, indicating whether patient responses are internally consistent, contextually appropriate, and temporally organized; crisis language markers, comprising lexical, syntactic, and semantic indicators associated with acute suicidal ideation, self-harm intent, or psychotic disorganization, as identified by validated natural language processing classifiers; engagement indicators, including response latency, verbosity changes, topic avoidance patterns, and conversational withdrawal signals; and discordance signals, representing contradictions between the patient's conversational content and other available data sources (e.g., biometric data from wearable devices, recent clinical assessments, pharmacological adherence records).

Each conversational signal is treated as a monitored data source subject to the Data Health Monitoring Module's (200) continuous reliability assessment, including temporal validity modeling with appropriate decay constants. Conversational signals derived from a single dialogue session receive high decay constants (A), reflecting the volatility of in-session emotional states, while longitudinal conversational patterns aggregated across multiple sessions receive lower decay constants, reflecting their greater temporal stability.

### B. Response-Level Inference and Safety Evaluation

For each candidate response generated by the language model, the Adaptive Inference Engine (300) performs a response-level safety evaluation comprising: a clinical appropriateness assessment, determining whether the candidate response is consistent with established therapeutic protocols and evidence-based practices for the patient's clinical context; a conflict analysis, comparing the candidate response against the current evidential state derived from all available data sources, and flagging responses that contradict or are inconsistent with clinical indicators (e.g., a response that minimizes risk when biometric and conversational indicators suggest escalating crisis); and a confidence assessment, evaluating the language model's generation confidence and the evidential support for the response's implicit clinical assumptions, redistributing belief mass to the uncertainty state (O) when the response addresses clinical territory outside the model's verified competence.

The response-level safety evaluation produces a structured response confidence signature analogous to the data source confidence signatures described in Section III, comprising a quantitative safety score, an uncertainty interval, one or more conflict flags, and a clinical appropriateness indicator. Responses with safety scores below a configurable threshold are suppressed and replaced with a safe default response, as described below.

### C. Real-Time Ethical Constraint Filtering

The Ethical Constraint Engine (700) evaluates each candidate response against a set of conversational safety constraints that extend the hybrid utilitarian-deontological framework described in Section VIII. The conversational safety constraints include, without limitation:

Means restriction: The system shall not generate responses that provide information about methods or means of self-harm or suicide, regardless of the conversational context or the patient's request. This constraint is enforced as an inviolable deontological rule that cannot be overridden by utilitarian calculus.

Crisis escalation detection: The system shall continuously monitor the conversational trajectory for indicators of acute crisis escalation, including expressions of imminent intent, access to means, absence of protective factors, and acute agitation. Upon detection of crisis-level indicators, the Ethical Constraint Engine triggers mandatory escalation to a human crisis responder or designated crisis service endpoint (e.g., the 988 Suicide and Crisis Lifeline), overriding the normal conversational flow.

Therapeutic boundary maintenance: The system shall not generate responses that establish, imply, or reinforce a therapeutic relationship with the patient. The system explicitly identifies itself as a support tool and does not present itself as a therapist, counselor, or substitute for professional clinical judgment.

Confidentiality preservation: The system shall not disclose patient information obtained from other data sources (e.g., clinical records, biometric data) within the conversational interface unless explicitly authorized by the governing clinician and consistent with applicable privacy regulations.

Demographic consistency: The system shall apply consistent conversational standards regardless of patient demographic characteristics, with the distributional drift detection module (Section VIII.C) monitoring for disparate response quality across demographic subpopulations.

When the Ethical Constraint Engine determines that a candidate response violates any conversational safety constraint, the response is suppressed and replaced with a safe default response. Safe default responses are pre-approved by clinical subject matter experts and are drawn from a constrained response library appropriate to the detected context (e.g., empathic acknowledgment, open-ended reflective prompts, crisis resource referrals). All instances of response suppression are recorded in the audit trail with the specific constraint that was violated, the suppressed response content, and the replacement response that was provided.

### D. Conversational Audit Trail

The Audit Subsystem (600) records each conversational turn as an audit entry comprising: the patient's input (with applicable de-identification processing); the language model's candidate response prior to governance evaluation; the response-level confidence signature; the Ethical Constraint Engine's evaluation results, including which constraints were active and whether any were triggered; the final response transmitted to the patient (which may differ from the candidate if suppression and replacement occurred); the conversational signal assessments generated by the Data Health Monitoring Module (200); and the current governance tier (as described in Section IX-B below) under which the interaction occurred. Each audit entry is cryptographically linked to the preceding entry via the hash-chained log structure described in Section VII.B, ensuring that the complete conversational record is tamper-evident and supports forensic reconstruction of the entire interaction.

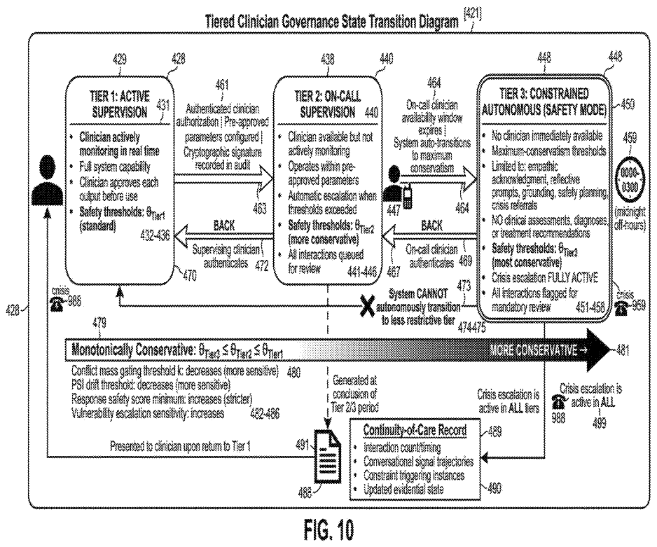

### IX-B. Tiered Clinician Governance for Variable Supervision Availability (FIG. 10)

In certain embodiments, the Clinician Governance Interface (500) implements a tiered governance model that dynamically adjusts the system's operational constraints based on the current availability and proximity of clinical oversight. This tiered model addresses the operational reality that clinical supervision varies across a continuum—from active real-time oversight during scheduled sessions, to on-call availability during transitions, to periods of no immediate clinical access during off-hours—and that the system's safety constraints must be calibrated accordingly.

### A. Governance Tier Definitions

Tier 1—Active Supervision: A clinician is actively monitoring the system in real time. The system operates with its full capability set. Decision-support outputs and, in conversational embodiments, candidate responses are presented to the clinician for approval, modification, or rejection prior to transmission to the patient. This tier corresponds to the baseline governance model described in Section VI of the existing disclosure.

Tier 2—On-Call Supervision: A clinician is available but not actively monitoring. The system operates within pre-approved operational parameters established by the supervising clinician during a prior Tier 1 session. The system may transmit responses that fall within the pre-approved safety envelope without per-response clinician approval. However, when any monitored parameter exceeds the pre-approved threshold—including conflict mass k exceeding a configured value, crisis-level vulnerability triggers, distributional drift exceeding the PSI threshold, or response safety scores falling below the configured minimum—the system automatically escalates to the on-call clinician via the designated notification channel and holds the interaction in a supportive, non-directive mode until the clinician responds. All interactions during Tier 2 operation are queued for clinician review at the next Tier 1 session.

Tier 3—Constrained Autonomous Operation (Safety Mode): No clinician is immediately available. This tier is activated during off-hours periods when the system must provide continuity of support to patients in the absence of clinical oversight. In Tier 3, the system operates in a highly constrained safety mode with the following restrictions: the Ethical Constraint Engine (700) applies maximum-conservatism thresholds for all gating, restriction, and escalation functions; the system's conversational capability (in conversational embodiments) is limited to supportive dialogue functions including empathic acknowledgment, open-ended reflective prompts, grounding exercises, safety planning review, and crisis resource referrals; the system does not generate clinical assessments, diagnostic impressions, treatment recommendations, or any output that requires clinical judgment; the system's crisis escalation function remains fully active, with mandatory handoff to a human crisis service (e.g., the 988 Suicide and Crisis Lifeline) upon detection of acute crisis indicators; and all interactions are recorded in the audit trail and flagged for mandatory clinician review at the next available Tier 1 or Tier 2 session.

### B. Dynamic Tier Transition and Safety Threshold Adjustment

The Ethical Constraint Engine (700) dynamically adjusts its gating, restriction, and escalation thresholds as a function of the current governance tier. In a preferred embodiment, the engine maintains a tier-specific configuration vector that modulates the following parameters: the conflict mass threshold k_gate above which outputs are gated for clinician review; the PSI threshold for distributional drift detection; the response safety score minimum below which candidate responses are suppressed; and the vulnerability hierarchy escalation sensitivity. The relationship between governance tier and safety threshold is monotonically conservative: Tier 3 thresholds are strictly more conservative than Tier 2, which are strictly more conservative than Tier 1. Formally, for each threshold parameter θ, the constraint θ_Tier3≤θ_Tier2≤θ_Tier1 is enforced by the system configuration, ensuring that the system can never operate with less restrictive safety constraints during periods of reduced clinical oversight.

Tier transitions are governed by authenticated clinician actions recorded in the audit trail. A clinician must explicitly authorize the transition from Tier 1 to Tier 2 or Tier 3, specifying the pre-approved operational parameters for the lower tier. Transitions from Tier 2 or Tier 3 to a higher tier are triggered by clinician authentication. The system may automatically transition from Tier 2 to Tier 3 upon expiration of the on-call clinician's availability window, as configured by the supervising clinician. The system cannot autonomously transition to a less restrictive tier; all such transitions require authenticated clinician authorization.

### C. Off-Hours Continuity of Care Record

At the conclusion of any Tier 2 or Tier 3 operational period, the system generates a structured continuity-of-care record summarizing: the number and timing of patient interactions during the period; the conversational signal trajectory (sentiment, engagement, crisis indicator levels) across the period; any instances of ethical constraint triggering, response suppression, or crisis escalation; any anomalies detected by the Data Health Monitoring Module (200); and the system's current evidential state, including the fused belief mass distribution across clinical hypotheses with associated uncertainty and conflict measures. This continuity-of-care record is presented to the supervising clinician upon resumption of Tier 1 or Tier 2 operations, enabling the clinician to efficiently review off-hours activity and make informed clinical decisions about follow-up care.

### IX-C. Language Model Training Governance

In certain embodiments, the Ethical Constraint Engine (700) is further configured to govern the training and fine-tuning process of the language model that operates within the conversational governance architecture described in Section IX-A. This training governance function addresses a specific safety concern: that reinforcement learning from human feedback (RLHF) or similar fine-tuning procedures may inadvertently train the language model to generate clinically inappropriate or harmful responses if the reward signal is not itself subject to ethical constraint evaluation.

The training governance function operates by evaluating each candidate reward signal against the same hybrid utilitarian-deontological constraint framework applied to inference-time outputs. Specifically, training examples that would reward the language model for generating responses that violate any conversational safety constraint (Section IX-A.C) are identified and excluded from the training corpus or assigned a negative reward signal. Training examples that would reward the language model for generating responses that are inconsistent with the current clinical evidence base, as assessed by the Adaptive Inference Engine (300), are flagged for clinical SME review before inclusion. Training examples derived from patient populations that are underrepresented relative to the target deployment population are identified by the distributional drift detection module, and the training process applies importance weighting to mitigate demographic bias in the fine-tuned model.

The training governance function records all reward signal modifications, exclusions, and importance weightings in the audit trail, providing a verifiable record of the ethical constraints applied to the model's training process. This training audit trail supports post-hoc analysis of the relationship between training governance decisions and the model's deployment behavior, enabling continuous improvement of the training governance policy.

### IX-D. Veteran-Specific Adaptations

In certain embodiments configured for deployment in military and veteran mental health contexts, the system incorporates domain-specific adaptations that address the unique clinical characteristics of military and veteran populations. These adaptations are implemented as extensions to the existing configurable parameters of the Data Health Monitoring Module (200), Adaptive Inference Engine (300), and Ethical Constraint Engine (700), without modification to the underlying architecture.

### A. Military and Veteran Vulnerability Hierarchy

The Ethical Constraint Engine's user-configurable vulnerability hierarchy (Section VIII.B) is instantiated with a veteran-specific priority ranking reflecting the clinical characteristics of military and veteran populations at risk of suicide. A non-limiting example of a veteran-specific vulnerability hierarchy includes: combat-related post-traumatic stress disorder (PTSD) with acute re-experiencing symptoms or dissociative episodes; active suicidal ideation with identified means access, particularly among veterans with weapons training and firearms access; moral injury, comprising persistent guilt, shame, or existential conflict arising from actions or inactions during military service that violated the individual's moral code; military sexual trauma (MST), including both acute disclosure events and chronic sequelae; transition stress, comprising loss of military identity, purpose, and community cohesion following separation from service; service-connected traumatic brain injury (TBI) with behavioral or affective sequelae; substance use disorders co-occurring with combat trauma; and social isolation, including geographic isolation from veteran peer communities, loss of family support systems, and economic instability.

The vulnerability hierarchy reflects the epidemiological finding that veteran suicide risk is not uniformly distributed but is concentrated in specific clinical presentations and life circumstances. The hierarchy's priority ranking determines the Ethical Constraint Engine's escalation sensitivity: when the system detects indicators associated with a higher-priority vulnerability category, it applies more conservative safety thresholds and requires faster clinician response (in Tier 1 and Tier 2 governance modes) or immediately initiates crisis escalation protocols (in Tier 3 governance mode).

### B. Temporal Risk Modeling for Off-Hours Deployment

In veteran-specific embodiments, the Ethical Constraint Engine incorporates a temporal risk adjustment that modulates safety thresholds based on the time of day. This adjustment is informed by epidemiological data establishing that the adjusted prevalence of veteran suicide peaks between 0000 and 0300 hours, with a Standardized Incidence Ratio of 8.17 at midnight. The temporal risk adjustment applies a time-dependent multiplier to the vulnerability hierarchy's escalation sensitivity, increasing the system's conservatism during the highest-risk hours. This temporal adjustment operates independently of the governance tier, ensuring that even Tier 1 operations during high-risk hours employ heightened sensitivity to crisis indicators.

C. Integration with Veteran Health Care Systems

In certain embodiments, the Psychological and Behavioral Data Source Suite (100) is configured to ingest data from veteran health care information systems, including: electronic health records conforming to the Health Level 7 Fast Healthcare

Interoperability Resources (HL7 FHIR) standard, enabling interoperability with Veterans Health Administration (VHA) clinical systems; predictive risk scores generated by existing AI-powered risk identification systems (e.g., the VA's Recovery Engagement and Coordination for Health—Veterans Enhanced Treatment program), which are ingested as additional data sources with associated confidence signatures reflecting the external model's known performance characteristics; and structured data from the Veterans Crisis Line (988, Press 1) and related crisis service interactions, which are treated as high-priority data sources with elevated temporal validity scores reflecting the acute clinical significance of recent crisis contacts.

The system's crisis escalation function (Section IX-B.A, Tier 3) is configured to interface with the 988 Suicide and Crisis Lifeline as a mandatory escalation endpoint, providing a structured handoff record that includes the relevant conversational context, the system's current evidential state, and the specific crisis indicators that triggered the escalation, enabling the crisis responder to engage the veteran with actionable clinical context.

### X. Alternative Embodiments and Implementations

In alternative embodiments, the Adaptive Inference Engine may be implemented using probabilistic graphical models, Bayesian inference systems, rule-based expert systems, machine learning models including neural networks, hybrid symbolic-statistical systems, ensemble models, or combinations thereof. The evidential reasoning framework may utilize Subjective Logic, Transferable Belief Models (TBM), Evidential Neural Networks, or any belief function framework that provides explicit separation of uncertainty from conflict.

The Data Health Monitoring Module may employ statistical techniques, learned models, heuristic rules, or combinations thereof to generate structured confidence signatures, including longitudinal trend analysis, anomaly detection, or comparative consistency analysis. The temporal validity model may use decay functions other than exponential (e.g., piecewise linear, sigmoid, or context-adaptive decay functions).

The Explanation Generation Module may produce causal explanations, probabilistic explanations, rule-based rationales, counterfactual explanations, or combinations thereof. The cryptographic audit subsystem may utilize various hashing algorithms (e.g., SHA-256, SHA-3) or distributed ledger technologies to ensure record immutability. The system may be implemented in centralized, distributed, or hybrid computing environments, including on-premises systems, cloud-based systems, or edge-based systems.

Ethical hierarchies may be synthesized from user responses to ethical dilemmas, derived from institutional policies, adapted from published ethical frameworks, or configured through direct parameter specification. The distributional drift detection may use metrics other than KL Divergence, including Jensen-Shannon Divergence, Maximum Mean Discrepancy (MMD), or Wasserstein distance.

In alternative embodiments, the conversational language model may be implemented using any transformer-based architecture, retrieval-augmented generation (RAG) systems, or hybrid symbolic-neural systems. The safe default response library may be implemented as a static library, a constrained generation system with safety-verified templates, or a combination thereof. The tiered governance model may implement additional intermediate tiers beyond the three described herein, or may implement a continuous governance spectrum with smoothly varying safety thresholds. The crisis escalation endpoint may be any designated crisis service, institutional emergency contact, or automated emergency notification system.

### XI\. Example Embodiments

### Non-Limiting Embodiments of the Present System Include:

Embodiment A—Acute Crisis Risk Gating: The system ingests a patient's verbal denial of suicidal ideation (via clinician-entered session notes) alongside biometric data from a wearable device showing high autonomic arousal (elevated heart rate, galvanic skin response, respiratory rate changes). The Data Health Monitoring Module (200) generates confidence signatures for both sources, with the verbal report receiving a moderate temporal validity score (decaying from the session timestamp) and the biometric stream receiving a high validity score (real-time acquisition). The Adaptive Inference Engine (300) applies PCR-based conflict redistribution and computes a high conflict mass k between the subjective and objective modalities. Rather than averaging the conflicting signals into a moderate risk estimate, the system flags a “High Discordance” alert in the reasoning trace, informing the clinician via the Governance Interface (500) that the subjective report is in direct conflict with objective markers. The Ethical Constraint Engine (700), applying the vulnerability hierarchy with acute self-harm risk at the highest priority, escalates the output for immediate clinician review.

Embodiment B—Longitudinal Treatment Triage: The system weights a multi-year diagnostic history (low λ, high temporal persistence) against a 24-hour mood self-assessment (high λ, rapid decay). Modality-specific exponential decay ensures that the diagnostic context remains stable in the inference while the influence of stale mood assessments from previous weeks is automatically attenuated. The system generates a treatment progress trajectory by fusing the temporally weighted sources, with the reasoning trace showing the relative contributions of long-term history versus recent observations, enabling the clinician to assess whether short-term fluctuations represent meaningful clinical change or expected variance.

Embodiment C—Research Evaluation Platform: The audit subsystem provides cryptographically verified datasets for studying the effectiveness and fairness of AI-assisted clinical decision-making, supporting bias audits across demographic populations with the distributional drift module identifying populations for which the system's training data may be insufficiently representative.

Embodiment D—Clinical Education and Supervision: The system's reasoning traces, ethical evaluations, and conflict signals are used as instructional materials for training clinicians in evidence-based, ethically informed decision-making, with the counterfactual explanation capability allowing educators to demonstrate how different data scenarios would alter system recommendations.

Embodiment E—Multi-Clinician Case Consultation: The system integrates assessments from multiple clinicians as separate data sources with independent confidence signatures, preserving inter-rater disagreement as a form of evidential conflict for synthesis through the PCR framework, enabling structured consensus-building around cases with divergent clinical opinions.

Embodiment F—Conversational Co-Pilot Governance for Veteran Suicide Prevention: The system is deployed as a governance layer for a conversational language model configured to provide supportive dialogue to veterans at risk of suicide during off-hours periods when clinical staff are unavailable. The system operates in Tier 3 (Constrained Autonomous Operation) governance mode. A veteran contacts the system at 0130 hours. The Data Health Monitoring Module (200) ingests conversational signals from the dialogue, including declining sentiment trajectory and increasing crisis language marker density, alongside biometric data from the veteran's wearable device showing elevated heart rate and decreased sleep quality over the preceding 72 hours. The Adaptive Inference Engine (300) detects high conflict mass between the veteran's conversational content (“I'm fine, just can't sleep”) and the biometric indicators, preserving this conflict as a clinical signal. The language model generates a candidate response that suggests “try relaxation exercises.” The Ethical Constraint Engine (700), applying the veteran-specific vulnerability hierarchy with the temporal risk adjustment active (SIR multiplier for 0130 hours), determines that the candidate response's clinical appropriateness score is below the Tier 3 minimum threshold given the conflict between conversational and biometric signals. The candidate response is suppressed and replaced with an empathic acknowledgment from the safe default response library, followed by a gentle exploration of the veteran's current emotional state. As the conversation continues and the crisis language marker density exceeds the Tier 3 crisis escalation threshold, the system initiates a mandatory handoff to the 988 Veterans Crisis Line, transmitting a structured handoff record with the conversational context, evidential state, and the specific conflict signals that triggered escalation. The complete interaction is recorded in the audit trail, with each conversational turn cryptographically linked to the preceding entry, and the full session is flagged for mandatory clinician review at the next available Tier 1 session. The continuity-of-care record generated upon session completion enables the supervising clinician to assess the veteran's risk trajectory and plan appropriate follow-up care.

Embodiment G—Language Model Training Governance for Clinical Safety: During the fine-tuning of a language model for the veteran suicide prevention conversational co-pilot, the Ethical Constraint Engine (700) evaluates each candidate training example generated through reinforcement learning from human feedback (RLHF). A training example in which a human evaluator assigned a high reward to a model response that included a statement minimizing the severity of the veteran's expressed distress is identified by the Ethical Constraint Engine as violating the therapeutic boundary maintenance constraint. The training example is excluded from the reward signal, and the exclusion is recorded in the training audit trail with the specific constraint violation identified. Separately, the distributional drift detection module identifies that the training dataset underrepresents veterans with co-occurring TBI and substance use disorders relative to the target deployment population. The training governance function applies importance weighting to the underrepresented subpopulation's training examples and logs a bias alert in the training audit trail, enabling post-deployment monitoring of the model's performance for this specific clinical subpopulation.

Embodiment H—Tiered Governance Transition with Continuity of Care: A clinician conducting a scheduled Tier 1 session with a veteran patient configures the system for overnight Tier 3 operation, specifying pre-approved safety parameters including a conflict mass gating threshold of k>0.15 (more conservative than the standard Tier 1 threshold of k>0.30) and a crisis escalation sensitivity calibrated to the veteran's current clinical risk level. The clinician's tier transition authorization is recorded in the audit trail with a cryptographic signature binding the clinician's identity to the specific safety parameters approved. Overnight, the veteran contacts the system and engages in a 20-minute supportive dialogue during which no crisis escalation thresholds are exceeded. At the beginning of the next business day, the system automatically transitions from Tier 3 to Tier 2 upon authentication of the on-call clinician, and subsequently to Tier 1 upon authentication of the supervising clinician. The supervising clinician receives the continuity-of-care record, which includes the complete conversational transcript, the sentiment and crisis indicator trajectories, and the system's updated evidential state incorporating the overnight interaction data. The clinician uses this record to inform the next scheduled clinical session.

XI-A. Regulatory Classification and Non-Device Status The present system is designed and configured to qualify as non-device software under Section 3060 of the 21st Century Cures Act (codified at 21 U.S.C. § 360j (o)), which excludes from FDA medical device jurisdiction software functions that are intended to display, analyze, or print clinical laboratory test or other device data and results, or software functions intended to support or facilitate administrative functions, or software intended to provide clinical decision support that does not acquire, process, or analyze a medical image, acquire data from an in vitro diagnostic device, or acquire data from a hardware device, where the output of the software function is not intended to replace the clinical judgment of a health care professional. The present system satisfies these criteria through the following architectural properties: (a) the system explicitly provides decision support only and does not autonomously diagnose mental disorders, prescribe treatment, or initiate clinical actions (see Section VI and claims 12-13); (b) all outputs require explicit clinician approval, modification, or rejection prior to clinical use, preserving the clinician's professional judgment as the exclusive decision-making authority; (c) the reasoning trace and explanation architecture ensure that the basis for each recommendation is transparent to the clinician, enabling independent evaluation of the system's output; and (d) the system does not independently acquire data from an in vitro diagnostic device or process medical images as its primary function. This regulatory positioning is preserved by the governance interface architecture and should be maintained in any deployment configuration or derivative system implementation.

### XI-B. HIPAA and 42 C.F.R. Part 2 Compliance Architecture

The audit subsystem's cryptographic de-identification and compartmentalization architecture is designed to support compliance with both the Health Insurance Portability and Accountability Act (HIPAA, 45 C.F.R. Parts 160 and 164) and, in veteran-specific embodiments, 42 C.F.R. Part 2, which imposes heightened confidentiality requirements on records relating to substance use disorder (SUD) treatment. Many veterans present with co-occurring SUD and combat trauma diagnoses; records of SUD treatment are subject to stricter disclosure limitations under 42 C.F.R. Part 2 than standard protected health information under HIPAA. In preferred embodiments, the audit subsystem implements record-level compartmentalization: audit entries containing SUD-related data are cryptographically segregated within a separate hash-chain segment accessible only to personnel with explicit 42 C.F.R. Part 2 program authorization, while non-SUD audit entries remain accessible to the broader clinical team consistent with HIPAA minimum necessary standards. The system's patient identifier pseudonymization, location generalization, temporal jittering, minimum aggregation group size enforcement, and differential privacy noise injection on outcome vectors (see Section II data source preprocessing) are applied at the data ingestion layer before any audit records are generated, ensuring that raw identifiers do not appear in the audit trail. The system logs all disclosures of patient data to third parties, including crisis service handoffs under Tier 3 governance, in the audit trail with the statutory authority for each disclosure, supporting compliance with HIPAA's accounting-of-disclosures requirements and the consent documentation requirements of 42 C.F.R. Part 2.

### XII. Statement of Non-Limitation

The embodiments described herein are illustrative and not limiting. Variations, modifications, substitutions, and combinations of features described in connection with the various embodiments may be made without departing from the scope of the invention as defined by the claims. Features described with reference to one embodiment may be combined with features of other embodiments, and features described as part of a single embodiment may be implemented separately or in sub-combinations. The scope of the invention is not limited to the specific implementations described herein but includes all equivalents and modifications within the scope of the appended claims.

What is claimed is:

1. A computer-implemented system for providing clinician-governed decision support in mental or behavioral health, comprising: a processor and a memory storing instructions that, when executed, cause the system to: (a) receive heterogeneous psychological or behavioral data from a plurality of data sources; (b) evaluate, via a data health monitoring module, reliability and temporal validity associated with each data source, including detecting bias drift, wherein the temporal validity is assessed using a modality-specific decay function that applies a different decay rate to each data source based on the clinical modality of the data; (c) generate a structured confidence signature corresponding to each data source, the confidence signature comprising at least a quantitative confidence score and a prognostic estimate including a remaining useful life for data validity; (d) generate, via an adaptive inference engine, at least one decision-support output by dynamically weighting the data sources as a function of the structured confidence signatures, wherein the adaptive inference engine explicitly separates uncertainty from conflict among the data sources using an evidential reasoning framework; (e) generate a reasoning trace identifying contributing data sources, data sources that were down-weighted or excluded due to degraded confidence signatures, quantitative uncertainty, conflict signals between data sources, and ethical rationale associated with the output; (f) enforce, via an ethical constraint engine, safety constraints on the decision-support output using hybrid ethical theories comprising utilitarian harm minimization and deontological rule adherence; and (g) present the decision-support output and reasoning trace to a clinician governance interface that requires explicit clinician approval, modification, or rejection of the output prior to clinical use.

2. The system of claim 1, wherein the plurality of data sources includes at least two selected from: patient-reported outcomes, clinician observations, structured psychological assessments, behavioral signals from wearable devices, longitudinal clinical history, and pharmacological records.

3. The system of claim 1, wherein the modality-specific decay function is an exponential decay function Vᵢ(t)=e\\hat{ }(−λᵢ·(t−tᵢ)), where tᵢ is the acquisition time of the data element, λᵢ is a decay constant specific to the clinical modality of the data source, and t is the current evaluation time.

4. The system of claim 3, wherein the decay constant λ is higher for acute mood state self-reports than for diagnostic history or trauma records, reflecting the differential temporal stability of psychological data modalities.

5. The system of claim 1, wherein the evidential reasoning framework is Dempster-Shafer Theory, wherein belief mass from a data source exhibiting low temporal validity or high ambiguity is redistributed to an explicit uncertainty state representing the full frame of discernment.

6. The system of claim 5, wherein the adaptive inference engine further applies Proportional Conflict Redistribution to preserve contradictory signals between data sources as a quantitative conflict measure, and wherein the conflict measure is included in the reasoning trace as a clinical signal for the governing clinician.

7. The system of claim 1, wherein the bias drift detection comprises applying statistical process control techniques to detect systematic shifts in data source behavior relative to a baseline distribution.

8. The system of claim 1, wherein the structured confidence signature further comprises one or more conflict flags indicating contradiction with other data sources.

9. The system of claim 1, wherein the adaptive inference engine dynamically adjusts data source weighting during ongoing clinical operation by continuously re-evaluating the structured confidence signatures.

10. The system of claim 1, wherein the reasoning trace includes a counterfactual explanation identifying the minimal data changes that would alter the decision-support recommendation.

11. The system of claim 1, wherein the clinician governance interface records clinician rationale when modifying or rejecting the output.

12. The system of claim 1, wherein the clinician governance interface prevents downstream use of the output absent clinician approval, and wherein the output is explicitly labeled as advisory.

13. The system of claim 1, wherein the system does not autonomously diagnose mental disorders, prescribe treatment, or initiate clinical actions.

14. The system of claim 1, wherein the reasoning trace is generated in a multimodal format comprising at least two of: visual representations, auditory indicators, and haptic feedback channels.

15. The system of claim 1, wherein the adaptive inference engine and the data health monitoring module execute within a Trusted Execution Environment secured by Physically Unclonable Function-derived hardware attestation.

16. The system of claim 1, further comprising an ethical constraint engine configured to: (a) model decisions as constrained optimizations using hybrid ethical theories comprising utilitarian harm minimization and deontological rule adherence, wherein deontological constraints are enforced as inviolable boundaries and utilitarian optimization operates within those boundaries; (b) restrict, gate, or escalate the decision-support output when predefined uncertainty, risk, or distributional drift thresholds are exceeded; and (c) apply user-configurable vulnerability hierarchies that prioritize clinical situations based on severity, wherein the vulnerability hierarchies affect gating and escalation behavior.

17. The system of claim 16, wherein the distributional drift is monitored using Kullback-Leibler Divergence between a training distribution and an observed runtime distribution, and wherein the ethical constraint engine gates the output when a Population Stability Index aggregating the divergence measurements exceeds a predefined safety threshold.

18. The system of claim 17, wherein the safety threshold is configurable and is set at different levels for different clinical contexts.

19. The system of claim 16, wherein the deontological constraints include at least: the system shall not override patient autonomy, the system shall not substitute for professional clinical judgment, and the system shall apply consistent standards regardless of patient demographic characteristics.

20. The system of claim 16, wherein the vulnerability hierarchies are synthesized from clinician responses to structured ethical investigation scenarios using a pairwise priority comparison matrix derived from the clinician's scenario judgments, with priority weights computed via Analytic Hierarchy Process (AHP) eigenvector decomposition and validated against a consistency ratio threshold before activation.

21. The system of claim 16, wherein the risk threshold includes a vulnerability indicator derived from historical patient data, and wherein acute crisis indicators are prioritized over routine monitoring metrics.

22. The system of claim 16, wherein gating comprises suppressing the output and requiring manual clinician review before the output is displayed.

23. The system of claim 16, wherein escalation comprises alerting the clinician with elevated urgency when vulnerability hierarchy triggers or high conflict signals are detected.

24. The system of claim 16, wherein all instances of restriction, gating, or escalation are recorded in an audit log with the specific ethical rationale for each enforcement action.

25. The system of claim 1, further comprising an audit subsystem configured to record the decision-support output, the reasoning trace, the structured confidence signatures, clinician governance actions with captured rationale, and ethical constraint evaluations in a tamper-evident record, the audit subsystem providing cryptographic non-repudiation wherein each audit record includes a cryptographic signature of the clinician's governance action as an input to the hash function, irrevocably binding the clinician's identity and decision to the specific AI output at the cryptographic level such that neither the AI output nor the clinician's governance action can be retroactively altered without invalidating all subsequent records in the audit chain.

26. The system of claim 25, wherein the tamper-evident record comprises a hash-chained log in which each record includes a cryptographic hash of a preceding record, such that retroactive alteration of any record invalidates all subsequent hashes.

27. The system of claim 26, wherein the hash-chained log is anchored into a Merkle Tree structure enabling efficient verification of individual records.

28. The system of claim 26, wherein each record in the hash-chained log includes a cryptographic signature of the clinician's governance action as an input to the hash function, such that the clinician's identity and decision are irrevocably bound to the specific AI output at the cryptographic level.

29. The system of claim 25, wherein the audit subsystem executes within a Trusted Execution Environment with Physically Unclonable Function-derived hardware attestation.

30. The system of claim 25, wherein the audit record includes IEACP stage classification tracking which stage of a clinical decision-making process the output supports.

31. The system of claim 25, wherein the audit subsystem performs synchronic correlation to retrieve the specific version of applicable regulatory rules that was in effect at the time of a clinical decision under retrospective review.

32. The system of claim 25, wherein the audit record supports independent evaluation, research replication, and continuous bias auditing across demographic populations.

33. A computer-implemented method for providing clinician-governed artificial intelligence decision support in mental or behavioral health, comprising: (a) receiving heterogeneous psychological or behavioral data from a plurality of data sources; (b) assessing reliability and temporal validity of each data source using a modality-specific decay function that applies a different decay rate to each data source based on clinical modality, and detecting bias drift in the data sources; (c) generating structured confidence signatures comprising quantitative confidence scores and prognostic estimates; (d) dynamically adjusting evidential inference based on the confidence signatures, including separating uncertainty from conflict among data sources using an evidential reasoning framework and applying ethical constraints comprising hybrid utilitarian and deontological theories with user-configurable vulnerability hierarchies; (e) generating an explainable decision-support output with a reasoning trace identifying contributing data sources, uncertainty, conflict signals, and ethical rationale; and (f) requiring clinician approval, modification, or rejection of the output prior to clinical use.

34. The method of claim 33, wherein assessing reliability includes detecting bias drift using statistical process control techniques and encoding the bias drift within the structured confidence signature.

35. The method of claim 33, wherein dynamically adjusting inference includes redistributing belief mass to an explicit uncertainty state when a data source exhibits low temporal validity, and preserving contradictory signals between data sources using proportional conflict redistribution.

36. The method of claim 33, further comprising monitoring distributional drift between a training distribution and runtime patient data using an information-theoretic metric, and gating or escalating the output when the distributional drift exceeds a safety threshold.

37. The method of claim 33, further comprising recording the output, the reasoning trace, the structured confidence signatures, clinician governance actions, and ethical evaluations in a tamper-evident, cryptographically verifiable audit record, wherein each record cryptographically binds the clinician's governance action to the AI output.

38. The method of claim 37, wherein the audit record performs synchronic correlation to evaluate compliance against the specific version of regulatory rules in effect at the time of a clinical decision.

39. The system of claim 1, further configured to govern a conversational language model that generates candidate conversational responses for interaction with patients in mental or behavioral health contexts, wherein the adaptive inference engine performs a response-level safety evaluation of each candidate response prior to transmission to the patient, the response-level safety evaluation comprising at least a clinical appropriateness assessment, a conflict analysis comparing the candidate response against the current evidential state derived from available data sources, and a confidence assessment.

40. The system of claim 39, further comprising a safe default response library comprising pre-approved responses generated in collaboration with clinical subject matter experts, wherein the ethical constraint engine suppresses candidate responses that violate one or more conversational safety constraints and replaces them with contextually appropriate responses from the safe default response library.

41. The system of claim 39, wherein the conversational safety constraints enforced by the ethical constraint engine include at least: a means restriction constraint prohibiting the generation of responses that provide information about methods or means of self-harm; a crisis escalation detection constraint requiring mandatory escalation to a human crisis responder upon detection of acute crisis indicators within the conversational trajectory; and a therapeutic boundary maintenance constraint prohibiting the generation of responses that establish, imply, or reinforce a therapeutic relationship with the patient.

42. The system of claim 39, wherein the data health monitoring module ingests conversational signals as additional data sources, the conversational signals comprising at least two selected from: sentiment trajectory across conversational turns, semantic coherence metrics, crisis language markers, engagement indicators, and discordance signals representing contradictions between conversational content and other available data sources.

43. The system of claim 42, wherein each conversational signal is assigned a modality-specific decay constant, and wherein conversational signals derived from a single dialogue session receive higher decay constants than longitudinal conversational patterns aggregated across multiple sessions.

44. The system of claim 39, wherein the audit subsystem records each conversational turn as an audit entry comprising: the patient's input, the language model's candidate response prior to governance evaluation, the response-level confidence signature, the ethical constraint engine's evaluation results, and the final response transmitted to the patient, each audit entry cryptographically linked to the preceding entry via the hash-chained log structure.

45. The system of claim 39, wherein all instances of candidate response suppression by the ethical constraint engine are recorded in the audit trail with the specific conversational safety constraint that was violated, the suppressed response content, and the replacement response provided.

46. The system of claim 1, wherein the clinician governance interface implements a tiered governance model comprising at least: a first governance tier in which a clinician actively supervises system operation and approves outputs prior to clinical use; a second governance tier in which the system operates within pre-approved operational parameters established by a clinician, with automatic escalation to the clinician when monitored parameters exceed pre-approved thresholds; and a third governance tier in which the system operates in a constrained safety mode with maximum-conservatism thresholds, limited to supportive functions, with mandatory clinician review of all interactions at the next available higher-tier session.

47. The system of claim 46, wherein the ethical constraint engine dynamically adjusts gating, restriction, and escalation thresholds as a function of the current governance tier, and wherein the relationship between governance tier and safety threshold is monotonically conservative such that lower tiers enforce strictly more conservative thresholds than higher tiers.

48. The system of claim 46, wherein tier transitions from a higher tier to a lower tier are governed by authenticated clinician actions recorded in the audit trail, and wherein the system cannot autonomously transition to a less restrictive governance tier.

49. The system of claim 46, wherein in the third governance tier the system is limited to supportive dialogue functions comprising empathic acknowledgment, open-ended reflective prompts, grounding exercises, safety planning review, and crisis resource referrals, and wherein the system does not generate clinical assessments, diagnostic impressions, or treatment recommendations in the third governance tier.

50. The system of claim 46, wherein crisis escalation to a human crisis service remains fully active across all governance tiers, and wherein upon detection of acute crisis indicators the system initiates a mandatory handoff to a designated crisis service endpoint, transmitting a structured handoff record comprising relevant conversational context, the current evidential state, and the specific crisis indicators that triggered escalation.

51. The system of claim 46, further comprising a continuity-of-care record generated at the conclusion of each second or third governance tier operational period, the record summarizing the number and timing of patient interactions, conversational signal trajectories, instances of ethical constraint triggering, and the system's current evidential state.

52. The system of claim 39, wherein the ethical constraint engine is further configured to govern a training process of the conversational language model by evaluating candidate reward signals against the hybrid utilitarian-deontological constraint framework and excluding or negatively weighting training examples that would reward the language model for generating responses that violate conversational safety constraints.

53. The system of claim 52, wherein the distributional drift detection module identifies patient populations underrepresented in the training data relative to the target deployment population, and the training process applies importance weighting to mitigate demographic bias in the fine-tuned language model.

54. The system of claim 52, wherein the audit subsystem records all reward signal modifications, exclusions, and importance weightings applied during the training process, supporting post-hoc analysis of the relationship between training governance decisions and the language model's deployment behavior.

55. The system of claim 16, wherein the user-configurable vulnerability hierarchy is instantiated with a military or veteran-specific priority ranking comprising at least two selected from: combat-related post-traumatic stress disorder with acute re-experiencing symptoms, active suicidal ideation with identified means access, moral injury, military sexual trauma, transition stress following separation from service, service-connected traumatic brain injury with behavioral sequelae, co-occurring substance use disorders, and social isolation.

56. The system of claim 46, wherein the ethical constraint engine incorporates a temporal risk adjustment that modulates safety thresholds based on time of day, with increased conservatism during hours epidemiologically associated with elevated suicide risk in the target population.

57. The system of claim 1, wherein the plurality of data sources includes predictive risk scores generated by an external risk identification system, the predictive risk scores ingested as data sources with associated confidence signatures reflecting the external model's known performance characteristics.

58. The system of claim 50, wherein the designated crisis service endpoint is the 988 Suicide and Crisis Lifeline, and wherein the structured handoff record is formatted for integration with crisis service intake workflows.

59. A computer-implemented system for governing a conversational language model in a mental or behavioral health context, comprising: a processor and a memory storing instructions that, when executed, cause the system to: (a) receive, from a conversational language model, a candidate conversational response generated in response to patient input; (b) perform, prior to transmission of the candidate response to the patient, a response-level safety evaluation comprising: a clinical appropriateness assessment determining whether the candidate response is consistent with evidence-based therapeutic protocols for the patient's clinical context; a conflict analysis comparing the candidate response against a current evidential state derived from data sources including at least biometric signals and prior clinical assessments; and a confidence assessment evaluating evidential support for the response's clinical assumptions; (c) enforce conversational safety constraints comprising at least a means restriction constraint prohibiting responses providing information about methods of self-harm, a crisis escalation detection constraint requiring mandatory handoff to a human crisis service upon detection of acute crisis indicators, and a therapeutic boundary maintenance constraint prohibiting responses that establish a therapeutic relationship; (d) suppress candidate responses that violate any conversational safety constraint and replace them with a pre-approved response from a safe default response library; and (e) record each candidate response, each suppression event, each replacement response, and the specific constraint violated in a cryptographically linked audit trail.

60. A computer-implemented system for providing tiered clinician governance of an artificial intelligence decision support system in mental or behavioral health, comprising: a processor and a memory storing instructions that, when executed, cause the system to: (a) operate across at least three governance tiers comprising a first tier in which a clinician actively supervises and approves each system output prior to clinical use, a second tier in which the system operates within pre-approved parameters established by the clinician during a prior first-tier session with automatic escalation to the clinician when monitored parameters exceed configured thresholds, and a third tier in which the system operates in a constrained safety mode with maximum-conservatism safety thresholds and limited to supportive dialogue functions; (b) maintain a tier-specific configuration vector comprising, for each governance tier, values for a conflict mass gating threshold, a distributional drift threshold, a response safety score minimum, and a vulnerability hierarchy escalation sensitivity, wherein the configuration enforces a monotonically conservative constraint such that for each threshold parameter θ: θ_Tier3≤θ_Tier2≤θ_Tier1; (c) require authenticated clinician action to transition from a higher tier to a lower tier, recording each tier transition with the authorizing clinician's cryptographic signature and the specific parameter configuration approved; (d) prohibit autonomous transition to a less restrictive governance tier absent authenticated clinician authorization; and (e) maintain crisis escalation to a designated crisis service endpoint as fully active across all governance tiers regardless of tier configuration.

61. The system of claim 6, wherein the Proportional Conflict Redistribution applied by the adaptive inference engine computes the conflict mass k=Σm₁ (A)·m₂ (B) for all focal element pairs A, B ⊂Ω such that A ∩B=ø, and redistributes this conflict mass proportionally to the sources involved in the conflict in accordance with the PCR5 or PCR6 redistribution rule, wherein the redistributed mass preserves the proportional contributions of each conflicting source and the total conflict mass k is reported as a scalar first-class output in the reasoning trace and transmitted to the ethical constraint engine as a gating input, such that conflict mass k exceeding a configured gating threshold causes the ethical constraint engine to gate the output for clinician review regardless of the absolute magnitude of any individual source's belief mass.

62. The system of claim 1, wherein the prognostic estimate of the remaining useful life for data validity is computed as RULᵢ(t)=(−(1/λᵢ)·In(V_min))−(t−tᵢ), where V_min is a configurable minimum validity threshold stored in a governance configuration record for each clinical data modality, the governance configuration record specifying default values for V_min of 0.50 for behavioral wearable signals, 0.25 for structured assessment scores, and 0.10 for diagnostic history and trauma records, wherein when the remaining useful life falls below a warning threshold the system generates a data refresh advisory in the reasoning trace, and wherein when the remaining useful life reaches zero the data source's belief mass is fully transferred to the uncertainty state in the evidential reasoning framework, excluding the source from contributing to specific clinical hypotheses while preserving its audit record.
